(19) United States
(12) Patent Application Publication
(10) Pub. No.: US 2002/0006604 A1
(43) Pub. Date: Jan. 17, 2002

(54) ONE DIMENSIONAL COMPOUND ARRAYS AND A METHOD FOR ASSAYING THEM

(76) Inventor: ALAN W SCHWABACHER, SHOREWOOD, WI (US)

Correspondence Address: KAROLINE K M SHAIR, CHOATE HALL & STEWART EXCHANGE PLACE, 53 STATE STREET, BOSTON, MA, 02109

(21) Appl. No.: 09/253,153

(22) Filed: Feb. 19, 1999

Related U.S. Application Data
(60) Provisional application No. 60/075,629, filed on Feb. 21, 1998.

(51) Int. Cl. C12Q 1/00; C12Q 1/70; C12Q 1/68; G01N 33/53

(52) U.S. Cl. 435/4; 435/6; 435/7.1; 435/7.2; 436/501; 436/518; 436/528; 436/530

(57) ABSTRACT

A method for the solid-phase synthesis of combinatorial libraries on a one-dimensional support, such as a thread, is provided. The method involves the cyclic permutation of structural features along the thread, in such a way that different structural features are repeated at a characteristic fixed frequencies along the thread. The thread is processed so as to generate a signal proportional to the activity of the compounds in the library, and the thread is then assayed by being drawn through an appropriate detector. The resulting time-domain signal is processed by Fourier transformation. Spikes in the frequency domain of the processed signal indicate the frequency at which structural features that contribute to the activity were created on the thread.

### PRIORITY INFORMATION

This application claims priority to the co-pending provisional application No. 60/075,629, entitled “One Dimensional Chemical Compound Arrays and a Method for Assaying Them” filed on Feb. 21, 1998, which is incorporated in its entirety by reference.

### GOVERNMENT SUPPORT

The present research was supported by a grant from the National Science Foundation (Grant Number CHE-9726030).

### BACKGROUND OF THE INVENTION

Combinatorial libraries have become important tools for the identification of compounds with desirable properties, both for practical purposes, such as the discovery of useful compounds like drugs, catalysts and other materials, and to answer other scientific questions (Geysen et al., Molec. Immunol. 1986, 23, 709-715; Houghton et al., Nature, 1991, 354, 84-86; Frank, R., Tetrahedron, 1992, 48, 9217-9232; Bunin et al., Proc. Natl. Acad. Sci. USA 1994, 91, 4708-4712; Thompson et al., Chem. Rev. 1996, 96, 555-600; Keating et al., Chem. Rev. 1997, 97, 449-472; Gennari et al., Liebigs Ann./Recueil, 1997, 637-647; Reddington et al., Science 1998, 280, 1735-1737). In general, the field of combinatorial chemistry encompasses the preparation of libraries of chemical compounds that are produced by reactions in which any of a number of species is attached to a number of intermediates at each step, yielding by their combination a much larger number of products. Such combinatorial synthesis approaches are widely recognized as important to a variety of tasks including pharmaceutical lead compound identification and development, and sensor and catalyst development (see, for example, Lam, K. S.; Lebl, M.; Krchnak, V. Chem. Rev. 1997,97, 411-448; Nefzi et al., Chem. Rev. 1997,97, 449-472; Gennari et al., Liebigs Ann./Recueil 1997, 637-647; Gravert et al., Chem. Rev. 1997, 97, 489-509; Thompson et al., Chem. Rev. 1996, 96, 555-600; Accounts Chem. Res. 1996,29 (Special Issue on Combinatorial Chemistry); Pirrung et al., Chem. Rev. 1997, 97, 473-488; Czarnik, A. W., Curr. Opin. Chem. Biol., 1997, 1, 60).

Two general approaches have been used to identify interesting substances from the numerous compounds resulting from combinatorial syntheses: deconvolution (see, for example, Geysen et al., Molec. Immunol. 1986, 23, 709-715; Houghton et al., Nature 1991, 354, 84-86) and encoding (see, for example, Czamik, A. W. Proc. Natl. Acad. Sci, USA 1997, 94, 12738-12739). In the deconvolution approach, a large number of compounds is prepared such that the compounds are grouped into pools, the activity of which are determined. The pools with the highest activities are resynthesized so as to divide the components further, and these smaller pools are iteratively tested and subdivided until individual compounds are identified.

The encoding approach involves associating each compound in the library with an identifier, in the form of a code or tag, then screening the full library of compounds. After those members with desirable properties are selected, the identifier is used to determine the identity of the hits. The identifier may be the spatial location of the compound in the library (e.g., a particular well in a microliter plate), or a readily identifiable chemical or other tag physically or spatially associated with the compound.

Each of these approaches has many variants, each with advantages and disadvantages; the preferred choice depends on the application. Deconvolution approaches are experimentally simple, can be carried out using assays for activity in solution, and allow analyses of pooled data derived that can lead to useful structure-activity generalizations. Disadvantages of deconvolution include the requirement of repetitive synthesis, complications associated with the analysis of mixtures (as when agonists and antagonists are present), and, most significantly, loss of information, as when a pool containing a single that a very high activity species and many low average activity species cannot be distinguished from a pool containing many members of moderate activity. Examples are known of substituents that diminish binding individually but combine to enhance binding, which validates this concern (see, for example, Liang et al., Science 1996, 274, 1520).

The encoding approach has the advantage that individual species are tested, so it is precise. Furthermore, encoding approaches are often amendable to robotic separate synthesis which can lead to great flexibility in possible assays for activity. On the other hand, such robotic syntheses require a substantial initial investment, and the number of compounds that can be investigated is limited. Several important encoding schemes have been developed that are amenable to analysis of very large numbers of compounds. Chemical tagging (see, for example, Brenner et al., Proc. Natl. Acad. Sci. USA 1992, 89, 5381-5383; Ohlmeyer et al., Proc. Natl. Acad. Sci. USA 1993, 90, 10922-10926; U.S. Pat. No. 5,565,324) is very effective for finding the “best” compounds, but full library decoding is impractical, so much of the library information is lost. Full library analysis is possible with spatially encoded libraries, among which the photolithographic “VLSIPS” approach provides very high information density (see, for example, Fodor et al., Science 1991, 251, 767-773; U.S. Pat. No. 5,143,854; U.S. Pat. No. 5,547,839), but requires sophisticated equipment and is substantially more elaborate than other procedures; the partially sequential nature of the synthesis best suits such systems to applications involving the smallest possible number of reagents at each stage, as in the synthesis of arrays of oligonucleotides. (see, for example, Array of oligonucleotides on a solid substrate, U.S. Pat. Nos. 5,445,934 and 5,510,270; Synthesis and screening of immobilized oligonucleotide arrays, U.S. Pat. No. 5,510,270; Printing molecular library arrays using deprotection agents solely in the vapor phase, U.S. Pat. No. 5,599,695). Other encoding systems include spot synthesis derivatization (Frank, R., Tetrahedron, 1992, 48, 9217-9232), which is simple but inherently serial rather than parallel, very labor intensive, and has a low information density.

The simplicity of the chemical tagging approach is partly due to the split/mix synthetic technique (see, Furka et al., Int. J. Pept. Prot. Res. 1991, 37, 487-493), in which solid-phase synthesis is carried out such that after each reaction performed on a subset of the support, the particles are remixed and subdivided for the next step. This results in ratios of compounds which are less dependent on reactivity rates than are found for synthesis carried out with mixtures of reagents, and each particle of solid support has a single synthetic history, so that activity at a specific particle implies activity of a specific compound. Furthermore, small particles, or beads, of solid support may be handled as suspensions in ordinary glassware, allowing synthetic procedures more closely approximating familiar organic synthetic techniques. Partly for this reason, a larger range of chemical reactions on a solid support have been carried out on such beads than on other types of support. (see, for example, Process for the simultaneous synthesis of several oligonucleotides on the solid phase, U.S. Pat. No. 4,689,405)

There remains a need for an encoding technique that would incorporate combinatorial synthesis with the simplicity of the split/mix approach, allow full library decoding in a simple way, and be amenable to assay without sophisticated apparatus. The present invention approaches this ideal. It incorporates the simplicity of the split/mix synthesis with the full library decoding of the spatial array. It offers as a significant aspect a particularly direct way of processing the information derivable from the library to develop a quantitative structure/activity relationship (QSAR).

### SUMMARY OF THE INVENTION

The present invention recognizes the desirability of combining full scale parallel synthesis with full scale data analysis and thus provides novel methods for preparing assays of chemical compounds and methods of analyzing them.

In one aspect of the present invention compound arrays are synthesized by providing a thread or support having functional groups and subjecting said support to one or more sets of reaction conditions, wherein each set of reaction conditions or reagents cycles with a specific period along the support, and wherein each reaction condition or reagent in a particular set is identifiable as a function of a unique distance or time. In certain preferred embodiments, the support comprises a single material. In other preferred embodiments, the support comprises a composite support. In still other preferred embodiments, the support comprises a discontinuous synthesized support arrayed on a continuous structural material. Thus, according to the method of the invention, a linear array of compounds results, with each compound uniquely identified as a function of its distance or time.

In another aspect, the present invention provides a novel method for analyzing compounds in an array. In general, according to the method of the invention, the compounds in the array are assayed in order to detect those compounds having a specific desired activity, and the compounds in the array are subsequently transported, preferably, at a constant velocity, through an appropriate detector capable of detecting compounds having a specific desired activity. This linear arrangement of data results in a unique way to analyze data obtained. Because of the mode of synthesis described above, the identity of a particular fragment of a compound cycles with a repeat time determined by the period used for the reactants or conditions used. Thus, subsequent mathematical processing of the data by Fourier transformation reveals any structure/activity relationships.

### DESCRIPTION OF THE DRAWING

FIG. 1A depicts the spiral winding of the thread on a cylinder.

FIG. 1B depicts the division of the cylinder into three equal regions, and the treatment of each region with a different coupling agent.

FIG. 1C depicts the cylinder in cross-section, with the compound coupled to each region represented by “A”, “B”, and “C”.

FIG. 1D depicts the resulting thread in linear form, with the compound coupled to each region represented by “A”, “B”, and “C”.

FIG. 2A depicts the thread wound around a larger cylinder than was employed previously, the division of the cylinder into three equal regions, and the treatment of each region with three different coupling agents.

FIG. 2B depicts the cylinder in cross-section, with the newly added moieties represented by “D”, “E”, and “F”.

FIG. 2C depicts the resulting thread in linear form, with the compounds now coupled to each portion of thread represented by “AD”, “BE”, “CE”, “CF”, “AF”, etc.

FIG. 3 depicts a preferred embodiment in which cylinders having two different diameters are utilized, and wherein the divisions are placed at the same location for each cylinder resulting in non-overlapping regions.

FIG. 4 depicts the overall scheme of how a thread is read.

FIG. 5 depicts the modified audio cassette used for thread analysis.

FIG. 6 depicts a fluorescent cell.

FIG. 7 depicts the time-averaged data from analysis of a library.

FIG. 8 depicts the binding profile obtained from the Fourier transformation.

### DEFINITIONS

In addition to their common and technically specific definitions, the following terms are intended to further comprise the following meanings:

“Thread”: As used herein, “thread” is a substantially one-dimensional support which supports synthetically useful sites for the attachment of a chemical library. The thread may take the physical form of a monofilament, a braided or wound assembly of filaments, a tape, hollow tube, or the like. The thread may be of any material that provides adequate physical, chemical, and mechanical properties. Suitable materials may be, but are not limited to, cotton, polyamide, polyester, acrylic, teflon, glass, steel, KEVLAR, and the like. Examples of relevant properties are tensile strength, elastic modulus, and inertness to the anticipated chemical treatments. The thread itself may be chemically modified so as to permit attachment of library members, covalently or otherwise, or the thread may support a continuous or discontinuous solid phase support for synthesis, as for example a series of beads arrayed along the thread, a grafted polymer layer, or a gel phase coated upon or impregnated into the thread. Many methods of functionalizing various materials and surfaces for use as synthesis supports are known in the art.

“Region”: As used herein, “region” is a segment of the thread which is exposed to a pre-selected chemical reagent or condition at a pre-selected time. A given contiguous portion of thread may belong to a plurality of overlapping regions.

“Member”: As used herein, “member” is one of a plurality of chemical compounds which together form a chemical library. Each member will be produced within a contiguous portion of the thread, as a consequence of the sequence of chemical regents to which that portion of the thread has been exposed.

“Cyclic averaging”: As used herein, “cyclic averaging” is a method of noise reduction which takes advantage of a library which is duplicated two or more times, with all members in the same relative order. Signals from each library member are averaged with signals from each subsequent occurrence of that member. This process may also be used with a shorter cycle time to extract useful information as described below.

“Signal”: As used herein, “signal” is the measured property of each library member. Examples of signals may be, but are not limited to, fluorescence, fluorescence polarization, luminescence, radiation, absorption of radiation, electromotive potential, pH, enzyme activity, cell growth and the like. The intensity of the signal may be directly or inversely proportional to some desirable property for which the library is being assayed. Examples of such properties are binding affinity for a metal, protein, nucleic acid, or other substances of interest, catalytic activity, or biological activity. Generally, any known method of solid-phase assay may be adapted to the present invention. Certain liquid-phase assays may be adapted as well by processing a thread which has been saturated with the appropriate liquid reagents, or by transfer of library members from the thread.

### DETAILED DESCRIPTION OF THE INVENTION

Recognizing the desirability of combining the power of full scale parallel synthesis with full scale data analysis, the present invention provides methods for the synthesis of linearly organized compound arrays, and methods for their analysis. In general, the library arrays are synthesized by providing a thread or support having functional groups, and subjecting said support to one or more sets of reagents or reaction conditions, wherein each set of reagents or reaction conditions cycles with a specific period along the support and wherein each reagent or reaction condition in a particular set is identifiable as a function of a unique distance or time. Thus, according to the method of the present invention, a linear array of chemical libraries results, with each chemical compound uniquely identified as a function of its distance or time. Furthermore, the linearization achieved by the method of the present invention provides unique methods for assaying chemical compounds and for analyzing compounds in an array. In particularly preferred embodiments, these compounds are analyzed for structure/activity relationships.

Certain examples of inventive libraries and methods are presented below, however these are not intended to limit the scope of the present invention.

Preparation of Libraries of Compounds

As discussed above, in one aspect, the present invention provides arrays of chemical compounds organized in a linear fashion, and methods of making these linearly organized arrays. In general, these arrays are prepared by providing a support or thread having reactive groups and subsequently subjecting the support to a set of reaction conditions or reagents, wherein each of the reaction conditions or reagents cycles with a specific period along the thread, and wherein each individual reaction condition or reagent in the set is identifiable as a function of a unique distance or time. As one of ordinary skill in the art will realize, in order to generate more complex libraries of compounds, it is desirable to subject the support to more than one set of reagents or reaction conditions and it is also desirable to provide the maximum number of combinations possible for a given set of reagents or conditions. Thus, the support is ideally subjected to two or more sets of reaction conditions or reagents. In preferred embodiments each subsequent set of reaction conditions is cycled with a specific period along the support with respect to other sets. In certain preferred embodiments, the periods are obtained by winding a support or thread around a geometric template and then dividing the surface of the geometric template into regions across the direction of the thread. In other preferred embodiments, the periods are obtained by measuring specific distances or times with respect to the support or thread. According to the method of the invention, in one preferred embodiment, all combinations of compounds are equally represented, as long as appropriate thread lengths and periods are utilized. Alternatively, in another preferred embodiment, a library is designed to represent a subset by utilizing a contiguous support shorter than is necessary for a particular full library. Similarly, a library having duplicates could be designed by utilizing a solid support longer than necessary to produce a single copy of each library member.

As one of ordinary skill in the art will realize, the support or thread may comprise any material upon which an array of compounds may be synthesized or attached, and that provides the desired physical, chemical and mechanical properties. Specific examples of relevant properties include, but are not limited to, tensile strength, elastic modulus, and inertness to the anticipated chemical treatments. In certain embodiments, this support comprises simply one material. In other embodiments, this support or thread is a composite material, that is, comprises a combination of one or more materials in any possible form. Examples of particularly preferred materials for use single material or composite supports include, but are not limited to, cotton, polyamide, polyester, acrylic, teflon, glass, steel, KEVLAR, metal, and the like, or any combination of one or more appropriate materials.

As one of ordinary skill in the art will also realize, a wide range of composite supports may be utilized. Exemplary supports include, but are not limited to, a filament or tape of an inert material capable of serving as a synthetic solid support for another material. This second material could be made by an established procedure, for example by radiation graft polymerization of substituted styrene (see, for example, Berg, R. H. et al., J. Am. Chem. Soc. 1989, 111, 8024-8026). Another possible support includes a crosslinked gel layer coated on the structural support. Properties (crosslink presence and density, polarity, stability, identity and density of functional groups and linkers for attachment of molecules) of this synthetic support material can be matched to a given application. Preferred properties would certainly depend upon the reaction conditions appropriate to synthesis and cleavage, and whether the library members are to be assayed while attached to the support, or after cleavage from the support.

In another particularly preferred embodiment, the support comprises a discontinuous support characterized in that this support comprises a discontinuous synthetic support material along a continuous structural support. One of the significant advantages provided by this support is that subdivision of the solid support into regions during the synthesis would be simplified if diffusion of reagents from one domain or region to another were precluded. This ultimately would facilitate the use of inert atmospheres and a wider variety of reaction conditions and reagents in a synthesis. Additionally, the discrete regions of a discontinuous support are unambiguously distinguished and identified during analysis and synthesis, with less precision required for distance measurements. Removal of samples for various purposes is also simplified since an object, rather than a position, is specified. An example of a discontinuous support includes, but is not limited to, small beads of gel support attached to a stable thread.

In general, once a support is selected for use in the library synthesis, the library is formed by the addition of certain sets of reagents, or alternatively or additionally by subjecting the support to a specific set of reaction conditions, as generally described above. The identity of each set of conditions or reagents is encoded by its distance from a fixed point, such that each variable will cycle at a fixed repeat distance and thus provide information about compounds in the linear array.

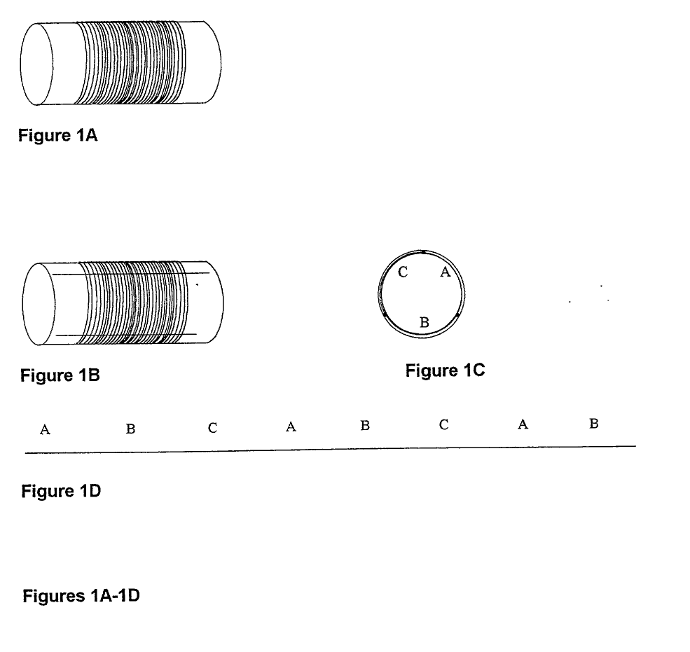

In order to more particularly describe a preferred embodiment of the present invention, the preparation of a library of compounds on a 1-dimensional thread support is described below. In general, in this embodiment, a library of compounds is prepared on a 1-dimensional solid support, or thread, in the following manner. The thread is wrapped around a cylinder in a single spiral layer as shown in FIG. 1A. As one of ordinary skill in the art will realize, other geometric templates can also be utilized, including but not limited to prisms of polygonal cross sections (e.g., hexagon templates, octagon templates, rectangular templates), cylinders with ridges to distinguish regions, flat plates, conic sections, and the like. Division of the surface of the cylinder lengthwise into a plurality of regions is followed by contacting each of the regions with a different reagent, as illustrated in FIG. 1B. Regions are preferably separated by use of an inert barrier or sealant, the sealant optionally being modified so as to emit or absorb light. The barrier is preferably an insoluble elastomer or wax-like material, such as a silicone or paraffin wax. Other techniques for separating or establishing regions include application of reagents without barrier, or division by solid walls forming channels between which liquid reagents may be passed, or masking for limited exposure to particles. This provides repeating domains on which are coupled each species, denoted in the scheme by letters and colors. The identity of each species is thus encoded by its distance from the end of the thread, such that each species to be coupled will cycle at a fixed repeat distance.

Other physical approaches to this goal are contemplated to be within the scope of the invention, such as printing reagents on a one-dimensional support using a wheel divided into regions. The reagent coupled to each region may consist of a single species, or may be a mixture of species so as to attach a mixture of moieties to the thread in that region.

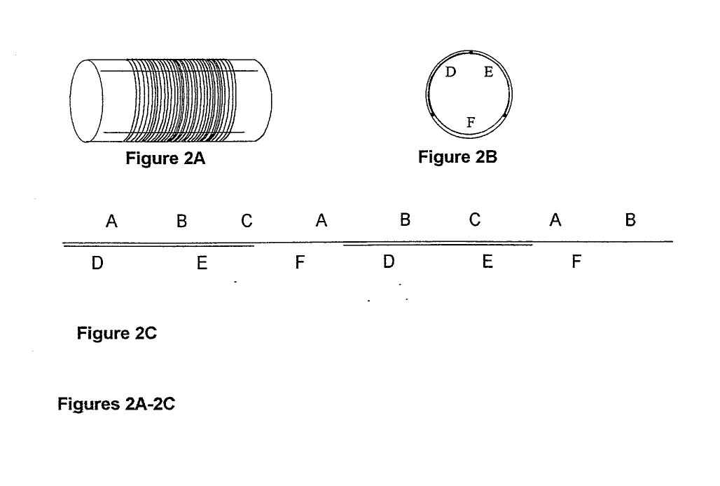

A second set of reagents is then coupled to the thread after the thread has been redivided into regions, preferably with a different repeat frequency, and preferably in such a manner that all reagent combinations are equally represented. This can be done by wrapping the thread around a second cylinder of an appropriate different diameter from the first, followed by division into regions, and coupling of the second set of reagents as depicted in FIGS. 2A-D. In the embodiment where reagents are applied by printing from a wheel, this corresponds to printing from a wheel of different diameter.

Repetition of these steps until all desired reagents have been used gives a library of compounds attached to the thread. All combinations can be equally represented, as long as appropriate cylinder ratios and sufficient linear solid support are used. Each different compound is uniquely specified by its location along the solid support. This is operationally analogous to the schemes which use a monolithic solid support, which is subdivided at each diversity generating step, in order to ensure that all combinations are represented with no duplicates (Stankova, et al., Pept. Res. 1994, 7, 292; U.S. Pat. No. 5,688,696). The distinction in the present invention is that the subdivision takes place in such a way that support is not physically fragmented, so information on species identity is retained spatially. This invention thus resembles the VSLIPS method, but with a one-dimensional rather than two-dimensional spatial encoding. Unlike the VLSIPS method, however, the method of this invention does not require expensive apparatus for synthesis or analysis of the library.

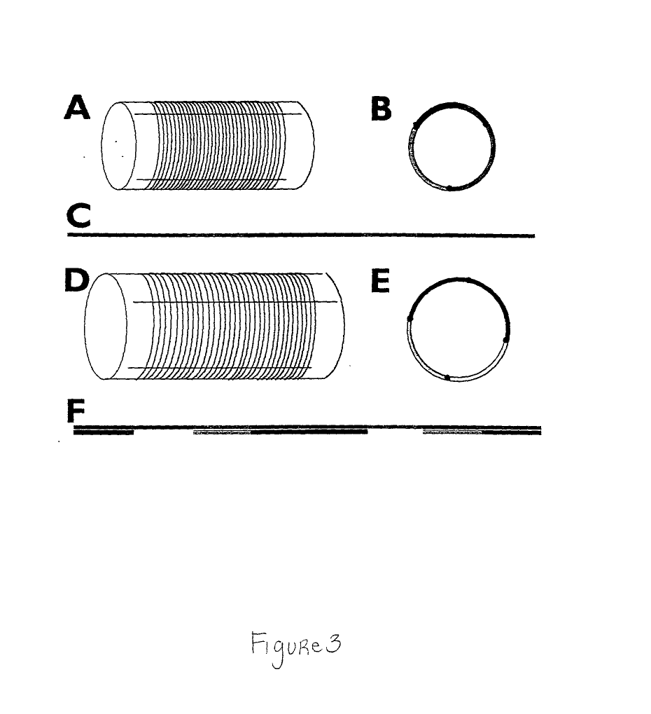

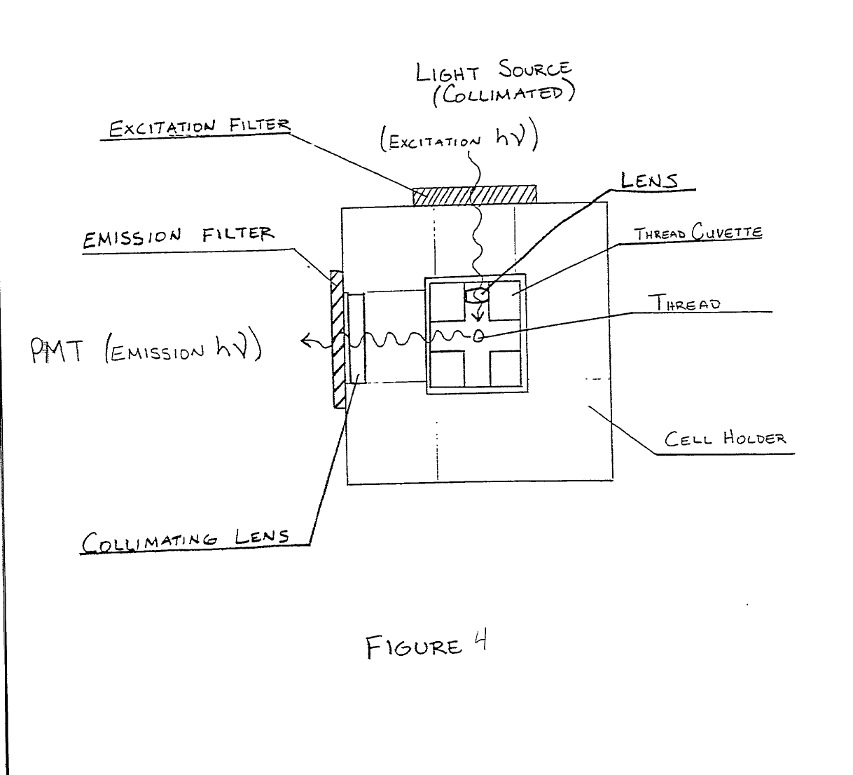

One particularly preferred embodiment of the invention is to place divisions between coupling regions at the same places on the thread for all cylinders, as shown in FIG. 3. While this is not required in general, it simplifies several aspects of the process, since all library components are of equal size and evenly spaced along the thread, and one copy of each combination appears before any repeat. Any barrier regions between library components are superposed for each cylinder, so that loss of usable solid support in these regions is minimized. The cost of this simplification is a limitation on the numbers of regions that can be used on each cylinder if all combinations are to be represented; the numbers must be relatively prime.

For example, if each library member is to occupy a length “L” of the thread, three reagents may be applied to the thread while it is wound on a cylinder of circumference 3L, five reagents may be applied while the thread is wound on a cylinder of circumference 5L, and seven reagents may be applied while the thread is wound on a cylinder of circumference 7L. The result of this process is a thread-supported library as follows:

circumference 3L cylinder: abcabcabcabcabcabcabcabcabcabcabcab . . .

circumference 5L cylinder: defghdefghdefghdefghdefghdefghdefgh . . .

circumference 7L cylinder: ijklmnoijklmnoijklmnoijklmnoijklmno . . .

As exemplified above, the first compound on the thread, “adi”, is not repeated until position 106, after all 105 possible combinations have been generated.

As one of ordinary skill in the art will realize, and as FIGS. 1A and 1B depict generally, other methods are also compatible with the inventive system, namely not all of the regions need be divided into the same size regions; rather some regions may be smaller than others. Additionally, the regions need not be divided at the same place, and thus overlapping regions can be utilized.

In yet another particularly preferred embodiment, as described generally above, the use of a geometric template such as a cylinder is not utilized; rather a set of reagents or reaction conditions is cycled at a specific repeat frequency and identified by its distance or time with respect to a support. In but one example, the synthesis of linear arrays of solid state materials can be prepared with useful emissive (E. Danielson et al., Nature 1997, 389, 944-948), magnetic (G. Briceno et al., Science 1995, 270, 273-275), catalytic (S. M. Senkan, Nature 1998, 394, 350-353), or conductive properties to name a few. These arrays could be prepared by vapor deposition or from soluble precursors, and could be made with compositions varying cyclically at a different period for each component. More specifically, it would be possible to vary, in a cyclic fashion, reagents, or other variables such as the temperature of a filament (for vapor deposition) or the concentration of the reactants (for soluble precursors).

Evaluation of Libraries for Activity

Clearly, one of the advantages to providing an array of chemical compounds is the ability to test these compounds for specific activities. In the present invention, the linear array of compounds can be subjected to a specific assay selected to distinguish compounds having a desired activity, and compounds having the desired activity can be identified by using an appropriate detector. In preferred embodiments, the linear array is moved through a desired detector and the identity of compounds is determined by their position on the array. Those of ordinary skill in the art will appreciate that position can be determined either by direct analysis of distance from a reference point, or by analysis of time for passage through the detector, where time is then related to distance, or through analysis of any other parameter that can similarly be related to distance. In particularly preferred embodiments, the array is passed through the detector at a constant rate, so that time is related linearly to distance. As will be discussed further below, this novel feature of the inventive system allows the analysis of specific assays and determination of structure/activity relationships.

As one of ordinary skill in the art will realize, assay of the library components for activity may be carried out in various known ways and may involve the detection of various activities such as binding activity, catalytic activity, inhibitor activity and promoter activity to name a few. Moreover, assay of certain library components may also be conducted while the compounds are still attached to the support or alternatively may be conducted after cleavage of the compounds from the support.

In but one example, detection of a bound analyte may be accomplished by measurement of emitted radiation or measurement of radiation absorbance. To identify those library members which bind to a particular analyte, for example a receptor, a tagged version of the receptor in solution may be contacted with the library, under conditions conducive to binding, and then visualized via the tag to determine where on the thread the receptor has bound and localized. Numerous procedures for identification of those sites bearing species that bind analyte are known to those skilled in the art (Kricka, L., Clin. Chem. 1994, 40, 347-357). In the present invention, identity of library members is uniquely encoded as a position along the thread. Particularly preferred embodiments are those wherein detection of analyte is accomplished by photometric methods, such as the detection of emitted light after irradiation or chemical treatment of the labeled library. The photometric method may measure or detect emission due to fluorescence, phosphorescence, or chemiluminescence of label. Colorimetric methods may also be employed, for example an ELISA assay for the bound analyte may be conducted, and the absorbance of light at an appropriate frequency may be detected in light reflected, scattered from, or passed through the thread.

The assay of compounds while attached to the thread need not be limited to sequential evaluation. Another preferred embodiment entails fully parallel assay by imaging while the thread is wrapped around a cylinder or other form. One example would be fully parallel evaluation of binding of a chemiluminescent tag, obtained by exposure of photographic film wrapped around a thread library, itself wrapped on a cylinder.

As mentioned above, the compounds prepared by the method of the present invention can also be cleaved off and assayed in solution. This could be carried out in pools, or as individual identified regions, by many procedures. If the linear solid support were cut into pieces, it could be treated as is any other solid synthetic support, with the distinction that one is aware of the identity of the library member on each region, so that information can be retained if desired. Examples of the use of the methods and arrays of the present invention in solution-based assays also includes the chemical cleavage of library members, but leaving these compounds within their synthetic solid support for storage and identification. This can be achieved for example, by using a non-extracting reagent, including, but not limited to light, hydrochloric acid or ammonia vapor, or by using a safety catch deprotection (see, for example, Panke et al., Tet. Lett. 1998, 39, 17-18; Hoffmann et al., “New Safety Catch Linkages for the Direct Release of Peptide Amides into Aqueous Buffers”, in R. Epton (Ed), Innovation and Perspectives in Solid Phase Synthesis and Combinatorial Libraries, pp. 407-410). After this procedure is applied, the thread library can be stored in this state. Subsequent wetting by an extracting solvent, (pH 7 aqueous buffer, for example) leads to solutions of library members confined to the region of the synthetic support. In this state, it is important to avoid contact of one region with another, to prevent contamination by diffusion. Either a support with discontinuous regions, or impermeable barriers, would serve to separate compounds along the thread. Several assays could then be applied.

In a particularly preferred embodiment, if the thread were embedded in or coated with an agarose gel matrix, cell-based assays could indicate which region of thread provides active compound ( see, Salmon et al., Mol. Div. 1996, 2, 57-63; Nestler et al., Bioorg. Med. Chem. Lett. 1996, 6, 1327-1330).

In other embodiments, the library members could be transferred to vessels for solution-phase assays, including, but not limited to the following examples. In one example, printing onto appropriate multiwell surfaces by contact with wetted solid support could be conducted. If the dry cleaved thread were wrapped on a cylinder with a small space between each region to avoid contact, wetted with a fine mist, incubated if necessary, and then rolled onto a flat or multiwell surface, spots of each library member would be formed, each in a separate well of known position. In another example, moving the thread across a perpendicular pulsed liquid stream would allow the liquid to extract and deliver library members to an appropriate vessel. Of course an array of pulsed streams could transfer a series of compounds, and then the thread could be advanced to allow the next series, transferring a row of compounds at a time to some kind of multiwell plate. Finally, in yet another example, the thread or support could be cut into regions and each placed in turn in an appropriate vessel.

The inventive system also, in another aspect, provides a method for assaying specific activities of compounds on an optical fiber support. In particular, the system utilizes a chemically derivatized surface optical fiber as the desired support for the synthesis of linear arrays of compounds. Specifically, such a library could be probed for binding to a fluorescent species in solution without rinsing because excitation by light constrained to the interior of the fiber by total internal reflection (a standard mode for optical fibers) would not excite fluorophore in solution, but would excite molecules bound in close proximity to surface by evanescent wave. Surface derivatization of an optical fiber could be carried out by standard silanization techniques, or by coating with a polymeric support. Additionally, in a preferred embodiment, a polymeric surface coating on the optical fiber could be made by soaking fiber in monomer, and directing light down the fiber. The evanescent wave at the optical fiber surface could initiate polymerization at the surface, avoiding bulk polymerization of monomer.

The abovementioned examples are intended to present a few preferred embodiments of the present invention; however, the scope of the invention is not limited to these particular examples.

Methods of Analysis

As discussed above, another aspect of the invention is the recognition that a linear arrangement of compounds provides a method for the analysis of data provided for a set of chemical compounds. In general, utilizing this method, whatever signal is measured to evaluate library components is subsequently mathematically treated as a function of thread distance or a specific time interval. Individual signals in the distance dimension, arising from individual library members, can be measured and processed to evaluate each thread-bound library component, giving data equivalent to that of other spatial encoding methods. The cyclic variation in structure along the thread is particularly amenable to data analysis however, and this is yet another aspect of the present invention.

As one of ordinary skill in the art will realize, if signal regions corresponding to the period due to a particular cylinder's circumference are averaged, the cyclically averaged resulting signal is equivalent to that of a pooling scheme where pools are based on the reactions carried out while the thread is wound around that cylinder. The pooling strategy is critical to the success of a deconvolution scheme; indeed Geysen has advocated multiple preparations of a library by all possible pooling strategies in order to select the best. Averaging the signal output directly from the detector over each repeat time will provide the same information in the same form as would pooled synthesis by all pooling strategies.

Thus, one aspect of this invention is a novel and powerful way to analyze the data. By moving the thread through an appropriate detector, the distance dimension (position along the thread) is mapped into the time domain. The present invention provides for the Fourier transformation of the resulting signal, either directly from the detector or after pre-processing. The time domain detector signal will consist of an evenly spaced set of measurements, where all compounds are assayed in the order they appear along the thread. Because of the mode of synthesis, the identity of a particular fragment of a molecule cycles with a repeat time determined by the period used for the reaction to install that part of the molecule. After Fourier transformation, a frequency domain spike indicates that activity depends to a significant degree on something that cycles at that frequency. In the case where the support is wrapped around a geometric template, the feature of the molecule most important to the activity assayed is indicated by the biggest spike, at a frequency corresponding to the circumference of the cylinder about which the thread was wrapped while that feature was being created or attached. The relative significance of the variation represented in the library of other portions of the molecule is indicated by the intensities of signals at their characteristic frequencies. Thus the intensity of a frequency peak indicates the extent to which the assayed property depends upon a variation in the molecule created or installed in a reaction using the corresponding cylinder.

The identities and relative fitness of specific groups for a given position in a molecule may be easily extracted from the FT spectrum at the characteristic frequency and its harmonics, as described below. The FT spectrum is a compact representation from which valuable information may be derived, not least the extent to which library variation can be represented as a linear combination of effects. Thus, trends in the entire library data are immediately apparent from the FT spectrum of the library regardless of the number of data dimensions.

Moreover, mixtures of compounds can be used at any position rather than pure compounds. The general significance of variation in this position can then be determined, though with lower discrimination between variants. For example, amino acids for peptide synthesis can be grouped in terms of the amino acids' properties, such as hydrophobicity, charge, or volume, and the significance to binding of that property in a given position of a peptide can be determined.

Since the entire library is decoded, it is possible to determine if the structural modifications in two or more places are related in overall functionality. For instance, pairing of groups in two positions in a molecule can be a binding determinant, and will be apparent from the FT analysis. For example, if an amino acid coupled on a 3 compound cylinder provides a low level of functionality, as does one coupled on a 17 compound cylinder, but together they have a higher level of functionality, the FT analysis will give a signal at a repeat time of 51 compounds.

It will be appreciated that the Fourier transformation method of the present invention does not require that the library be prepared and assayed on a one-dimensional thread. Libraries prepared by VLSIPS, or arrayed in microliter plates, for example, can be assayed by appropriate methods, and the resulting data can be arranged in series such that selected structural features reappear at characteristic frequencies in the series. The data may then be treated as a time series of data points and subjected to Fourier transformation analysis as taught above. Furthermore, no solid support need be involved at all. Experiments on multiple drug interactions could be carried out by passing a dilute suspension of cells down a tube to which various sets of drugs are added in a cyclic way with different cycle times for each drug, and separated by air bubbles.

Advantages of the 1-D organization described herein stem from the power and versatility of representation of multiple dimensions of variability in frequency. Those of ordinary skill in the art will appreciate that the relevant useful principles can also be applied to data arrays of higher dimensionality. To give but one example, if one measured activity of a library by several assays, one could have a 2-D array, where one of the dimensions corresponds to structural variation, and the other to the type of activity. One could define as “signal” a function of all the assay outputs that would reflect selectivity as well as activity, and process the resulting 1-D array. A more flexible approach would be to use a 2-D FT, and then to deconvolute using a signal corresponding to the desired selectivity. Those of ordinary skill in the art will recognize other variations that similarly fall within the scope of the present inventive approach.

These approaches are also applicable to the design and preparation of sparse libraries, where the number of members is smaller than the total number of combinations possible with the parameters to be varied. When choosing which of the possible combinations to prepare, it is advantageous to consider representation of all possible combinations as a sequence of variants, with each structural or procedural variation cycling at a characteristic frequency along the sequence. The subset chosen for preparation should be a contiguous region of this representation of the possible combinations. This allows FT analysis of the library, regardless of the actual synthetic or encoding strategy, even without evaluation of all possible combinations. It also provides an optimally diverse set of variants (Freier et al., J. Med. Chem. 1995, 38, 344-352; Konigs et al., J. Med. Chem. 1996, 39, 2710-2719). If many parameters are varied, this scheme provides that all pairwise combinations of all members are represented in the subset.

Description of Library Preparation and Analysis

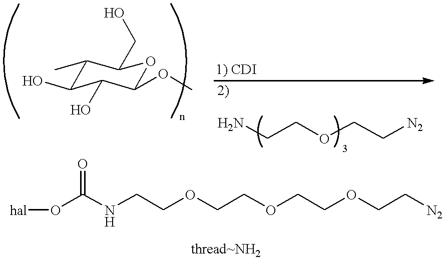

Cotton thread is an inexpensive, convenient, and appropriate solid support for peptide synthesis. In the example provided below, cotton thread was treated with carbonyldiimidazole, generation of the intermediate acyl imidazolide was confirmed by reflectance IR, and the functionalized thread was then subjected to reaction with 1,11-diamino-3,6,9-trioxaundecane. The resulting thread, with a urethane-linked oligoethyleneglycol terminated by an amine group is abbreviated herein as thread˜NH₂. A density of amine groups of 5×10⁻⁸ mol/cm was determined by ninhydrin assay (Stewart J. M.; Young, Solid Phase Peptide Synthesis; Pierce Chemical Co., 1984). Peptide coupling was carried out under conditions previously specified for peptide synthesis on a cellulose support, using FMOC protected HOBT esters at 0.3M in NMP (Frank, R. Tetrahedron 1992, 48, 9217-9232). Acylation was monitored by the bromophenol blue method during coupling (Krchnak et al., Collect. Czech. Chem. Commun. 1988, 53, 2542), and quantitated by ninhydrin assay in selected cases. (Ninhydrin monitoring was used when developing appropriate reaction conditions, but not during library synthesis, since it is a destructive method.) Successive 30 min. couplings of FMOC-Ala to thread˜NH₂, with acetic anhydride endcapping after each coupling before deprotection, gave successive yields of 50%, 70%, 100%, 100%. Thus some of the amino groups of the thread˜NH₂ appear to be less accessible than others. Three alanines were therefore coupled to the thread before library synthesis, to ensure that these less reactive groups were terminated in the same way throughout the thread.

Cylinders of ultra high molecular weight polyethylene (UHMW PE), were machined about 30 cm long, and of precise diameter so as to have circumferences of 3, 5, 7, 11, 13, 17, 19, 23, and 29 cm. A winding machine analogous to those used for winding electronic tuning coils was used to wrap thread very evenly in a single layer around these cylinders. Division lengthwise along the cylinder into regions onto which distinct amino acids were to be coupled was carried out as follows: a modified hot-melt glue gun was used to apply a paraffin wax barrier in parallel lines ruled every 1 cm lengthwise along the cylinder of thread. It is particularly advantageous if a black crayon is used as this wax, for reasons described below in the section on reading the library. The wax was applied in sufficient quantity that bleed through of peptide coupling reagents did not occur over the time of coupling. Solutions of NMP, HOBT, DIC, and FMOC amino acid at 0.3M were allowed to react for 30 min. to form activated ester, and then applied to the thread by pipette, being careful to keep each amino acid to its own space between wax lines. At this concentration, the amount of activated amino acid absorbed by the region of thread is sufficient to acylate the peptide, as has been observed with paper solid support. In some cases calorimetric assay allows qualitative assessment of the evenness and completeness of coupling. Over the course of the coupling reaction, bromophenol blue adsorbed on the thread changes from blue to yellow as the amine groups are consumed. Regions that remained blue were blotted to remove acylation solution, and recoupled in situ. After coupling, all regions were blotted and removed from the cylinder for endcapping, rinsing, deprotection with pyrrolidine, rinsing, bromophenol blue treatment, and then wrapping around the next cylinder for further reaction. At the end of the synthesis, sidechain deprotection was carried out with TFA in CH₂Cl₂. The most vigorous conditions needed were 50% TFA for 2 h. at room temperature. Under these conditions, cellulose is partially degraded and 50% of the peptide is lost from the thread (ninhydrin assay). For simple Boc removal 20% TFA for 20 min is sufficient, and causes little loss of material.

After preparation of the desired library, the assaying and analysis of the library was conducted. First, fluorescein-conjugated streptavidin was incubated with the thread library and those library components which bound to streptavidin became fluorescently labeled. Blocking and incubation procedures were used, similar to those commonly applied for immunoassays.

Wheels with sides to them to hold thread were installed in ordinary audio cassette cases, along with PTFE tubes to direct the thread which replaces the tape. This is a convenient embodiment, but spool size need not be limited to fit in an audio cassette. An ordinary audiotape player with the record/play heads removed acted to pull the thread at a constant rate, with the thread path being determined by the placement of the PTFE tubes. These tubes connect the cassette to a cell which was made to fit into a standard fluorescence spectrometer.

The thread was pulled at a constant rate through a monochromatic beam of light focused on the thread, and the dispersed light was filtered and then detected by a photomultiplier tube (PMT). The PMT signal was fed into a computer that recorded the time course of the signal. Time corresponds to distance along the thread because of the constant speed of the thread.

An aluminum block the size and shape of a standard fluorescence cell was prepared. Teflon tubes directed thread down the corner of the block, and up through the light beam in the center. A lens focused the excitation beam into a small spot (<1 mm) on the thread. In the embodiment exemplified here, lenses to pick up the emission were built into the spectrometer, but for use in a standard fluorescence spectrometer, a second lens would be installed in the cell to collimate emitted light.

A simple spectrometer was prepared with an aluminum cell holding block, with windows for lenses, filters, and the PMT. A quartz halogen light source was collimated, filtered through an interference filter (“excitation filter”), focused on the thread with lenses in the block and cell (focus adjustment was by external micrometer adjustment of the lamp). A collecting lens picked up emitted light, filtered it through a second interference filter (“emission filter”) mounted in front of the PMT (a stand alone unit from PTI, Inc.). Analog voltage output was run through an A/D board to a computer, and recorded using a simple BASIC program.

The data obtained from the thread reading was plotted on a graph using the data as single, discrete points plotted in arbitrary time units. This plot showed the overall signal of the entire library. There were regular peaks, as well as dips at regular intervals. These dips represent the areas where the black wax had been applied. For this embodiment, thread with small residual fluorescence was used to signal these dividing regions. Since there was a strip of black wax at each 1 cm interval, it was possible to merely count the peaks from the beginning of the library to find out which compound a particular peak represents.

Evenly spaced peaks were obtained by taking the average peak height above the average dip on either side. Each peak in the data represents a separate compound, and since the absolute beginning of the library is known, the identity of each peak was determined simply by measuring the distance from the end of the library.

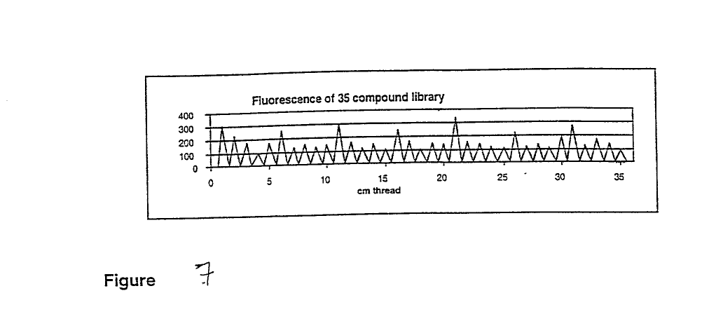

Since this particular library was known to repeat after 35 compounds, cyclic averaging over the appropriate repeat (peak 1 is averaged with 36, 71, etc...) was used to reduce noise and give a more reliable value for each peak height. The resulting plot of peak height vs. distance along the thread is presented in FIG. 7.

The peaks in FIG. 7, going from left to right, represent the following series, in this order:

A1, B2, C3, D4, E5, A6, B7, etc. . . .

Where:

| X1 | X2 |
|---|---|
| A = His | 1 = Ac Leu |
| B = Ser | 2 = Ac Phe |
| C = Asp | 3 = Bz |
| D = Ala | 4 = Ac |
| E = Phe | 5 = Ac His |
| | 6 = Ac Glu |
| | 7 = Ac Gly |

In this library, the expected highest peaks are those representing His in the final amino acid position (X₂-His-Pro-Gln-Phe-Ala-Ala-Ala-thread). The endcapping species should make less difference (Devlin et al., Science 1990, 249, 404-406; Lam et al., Nature 1991, 354, 82-82; Schmidt et al., J. Mol. Biol. 1996, 255, 753-766). Both of these expected results are seen. Profiles for group fitness at a given position may be obtained by cyclic averaging over appropriate shorter cycle times that correspond to a given cylinder.

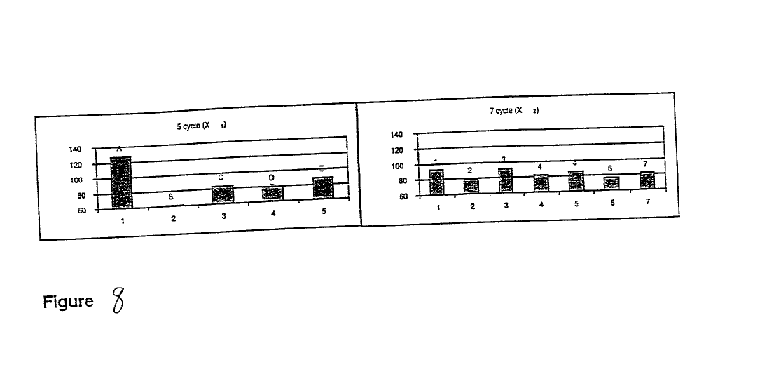

The data obtained from the thread reading was reduced to 2 points per compound, as outlined above (one point for each signal, taken as the average rise above the valley on either side of the signal, and one point between each peak). The Fourier transformation was done using a basic program using standard algorithms (Lynn et al., Introductory Digital Signal Processing with Computer Applications; Wiley: Chichester, 1989.; Press et al., Numerical Recipes in C: The Art of Scientific Computing; 2 ed.; Cambridge Univ. Pr.: Cambridge, 1993.; Blahut, R. E. Fast Algorithms for Digital Signal Processing 1985.; http://theory. Ics. mit.edu/˜fftw/; http://www.speech.cs.cmu.edu/comp.speech/Section 2/Q2.4.html). In a preferred embodiment, the FT should be resonant: a radix 2 algorithm is less appropriate, and would require oversampling of data. The “waveform” corresponding to efficacy of particular amino acids installed on a given cylinder was extracted as follows. The real and imaginary parts of the peak at the relevant frequency were extracted from the frequency domain, as were all harmonics. These values were then put into a smaller array and fourier transformed back to the time domain. The resulting “waveform” represents the output signal for each of the functional groups added on that cylinder. The signal for the 35 compound library, shown in FIG. 7, was Fourier transformed, and the waveforms corresponding to the 5 and 7 cm cylinders were extracted from the FT spectrum. These waveform binding profiles are shown in FIG. 8.

Experimental Detail

Preparation of amino functionalized cotton thread

A one dimensional cotton support was rinsed with 10% (v/v) HOAc/H₂O 15 times, each rinse being approximately 30 seconds at room temperature with 50 ml volume. This sample was then washed with distilled water 15 times, 10% NaHCO₃ 10 times, distilled water 10 times, EtOH 10 times, then CH₃CN 15 times. The thread was then dried by Soxhlet extraction with CH₃CN over CaH₂ under N₂. The thread was placed in 50 ml of a solution of 10.14 g CDI in 250 ml CH₃CN at room temperature for 24 hours under N₂ with shaking. The solution was checked by IR for the carbonyl peak at about 1670 cm⁻¹. When the peak disappeared, more CDI was added. Once the height of the peak stopped changing, the reaction had gone to completion. The thread was then rinsed with CH₃CN 10 times. The thread was placed in pure tetraethyleneglycol diamine at room temperature for 24 hours under N₂. The thread was then rinsed with 10% HOAc/H₂O 10 times, distilled water 15 times, saturated NaHCO₃ 10 times, distilled water 12 times, EtOH 10 times, and CH₃CN 15 times. Ninhydrin analysis yielded an amine concentration of 1.92×10⁻⁷ mol/cm.

Library Preparation

A small library of 35 peptides was prepared, as X₂-X₁-Pro-Gln-Phe-Ala-Ala-Ala-thread. H-Pro-Gln-Phe-Ala-Ala-Ala˜thread was prepared by couplings to the whole thread in a flask; only the X₁ and X₂ amino acids which constitute the library variation were added while the thread was wrapped around a cylinder. The thread was wrapped around the 5 cm circumference cylinder to couple X₁, which is chosen from (FMOC) His, Ser, Asp, Ala, Phe (denoted A-E respectively). After endcapping, deprotection, and wrapping around the 7 cm cylinder, X₂, chosen from Leu, Boc-Phe, Bz, Ac, His, Glu, Gly (denoted 1-7 respectively) was added. The Boc-Phe results in a free amine terminus, while the other amino acids, coupled as their FMOC derivatives, are N acetylated before binding studies. Fmoc deprotection and acetylation were followed by deprotection of sidechains in 50% TFA/DCM for 2 h. The library was rinsed thoroughly, blocked by incubation with 3% bovine serum albumin, and exposed to streptavidin-fluorescein conjugate. The thread was dried, and then read on the thread reader.

Coupling, Endcapping, and Deprotecting

Coupling: 3 m of thread-amine, prepared as described above, was rinsed with NMP 4 times, 7 ml each time. The Fmoc-amino acid esters were prepared by mixing 0.5 ml 1M Fmoc-amino acid in NMP with 0.5 ml HOBT/NMP and 0.5 ml 1.2M DCC/NMP solutions. This solution was allowed to react at room temperature for 60 min with vortexing. Activation was indicated by the production of a precipitate. The thread was placed in the Fmoc-amino acid HOBT ester solution at room temperature and the vial was shaken for 30 minutes.

Endcapping: The thread was rinsed with 2% Ac₂O/DMF solution 2 times, 7 ml each time. The thread was then quenched with 2% Ac₂O/1% DIEA/DMF at room temperature for 30 minutes and rinsed with DMF 4 times and CH₃CN 4 times, ˜7 ml each time.

Deprotecting: The thread was placed in 10 ml 20% Pyrrolidine/DMF solution at room temperature for 25 minutes. It was then rinsed with DMF 4 times and CH₃CN 4 times, ˜7 ml each time.

Final Deprotection: The final deprotection was carried out in 50% TFA in CH₂Cl₂ for 2 hours. When this procedure was complete, coupling efficiency was determined by ninhydrin analysis.

The results were as follows:

| | [amines] per | % Amine | % coupled |
|---|---|---|---|
| Sample | cm thread | remaining | this step |
| Blank thread | 1.02 × 10⁻¹⁵ | — | — |
| Amine linked thread | 5.93 × 10⁻⁸ | 100 | — |
| After endcapp., 1 coupling | 6.52 × 10⁻¹³ | 1.1 × 10⁻⁵ | (100) |
| After deprot., 1 coupling | 1.11 × 10⁻⁹ | 1.9 | 2 |
| After endcapp., 2 couplings | 9.04 × 10⁻¹⁴ | 1.5 × 10⁻⁶ | (100) |
| After deprot., 2 couplings | 9.30 × 10⁻¹⁰ | 1.6 | 84 |
| After deprot., 3 couplings | 6.56 × 10⁻¹⁰ | 1.1 | 71 |
| After deprot., 4 couplings | 7.13 × 10⁻¹⁰ | 1.2 | 109 |

Coupling on a Cylinder

The thread prepared above was rinsed in 0.1% Bromophenol Blue (BPB)/DMF for 2 minutes. It was then rinsed with EtOH 4 times and CH₃CN 4 times, each using 10 ml volume per 3 m thread. BPB was added to the thread, causing the thread to turn blue. The thread was wrapped around the selected cylinder. Black wax was applied to divide the cylinder into 1 cm sections such that these sections were sealed against liquid run-through. 1 m of thread was left at the beginning for connection into the thread reader. The first portion of the library was identified such that it will again be the first section used in the library. We coupled the Fmoc-Amino acid solutions, prepared as described above in the coupling step, were placed on each 1 cm wide section of the cylinder, at about 40(L per cm²). After 30 minutes, the thread turned from blue to a green-yellow color, signifying that the coupling was complete. At this point the excess solution was removed by blotting with an absorbent tissue and more coupling reagent was added. After 30 minutes, excess solution was removed and more activated amino acid was added for another 30 minutes.

At this point, the thread was removed from the cylinder. Endcapping and deprotection were carried out as described above with the entire thread immersed in reagent solution. Each subsequent coupling was carried out by again wrapping the thread on a specific sized cylinder.

Ninhydrin Test of Amine Concentration

Reagents A, B and C were made up as follows:

Reagent “A”: 10⁻²M KCN in H₂O, diluted to 100 ml in pyridine

Reagent “B”: 20.7 g Phenol in 5.0 ml EtOH

Reagent “C”: 0.50 g Ninhydrin in 10.0 ml EtOH

100 (L “A”, 50 (L “B”, and 25.0 (L “C” added to a vial. Amine sample was added to this vial. The vials were heated at 100 C. for 10 min. The vials were then quenched at 0 C. The solutions were then diluted with 3.0 ml of 60% EtOH/H2O. UV-Vis absorbance readings were taken at 570 nm.

Ninhydrin test was done of both thread samples and standard amine samples at the same time. The standard amine samples were made by diluting tetraethyleneglycol diamine in 60% EtOH/H₂O such that the final concentration of amine groups in solution ranged between 2×10⁻⁸ to 2×10⁻⁷.

Absorbance was plotted versus concentration in the case of the standard solutions and versus cm thread for the thread samples. Regression line was added to both plots. By dividing the slope of the line for the thread samples by the slope of the line for the standard solutions, the value of moles of amine per cm thread was obtained.

Streptavidin-Fluorescein Binding

The prepared thread library was rinsed with tris buffer (pH 7.4) 2 times with 10 ml volume. The thread was then immersed in 1% BSA in NaCl (0.15M)/ Tween 20 (0.04M)/ tris buffer (pH 7.4), and vortexed for 1.5 hours to block the non-specific binding sites on the thread. Streptavidin fluorescein conjugated stock solution (1 mg/ml, 0.020 ml) was added to this solution and vortexed for 1.5 hours.

Thread Reading

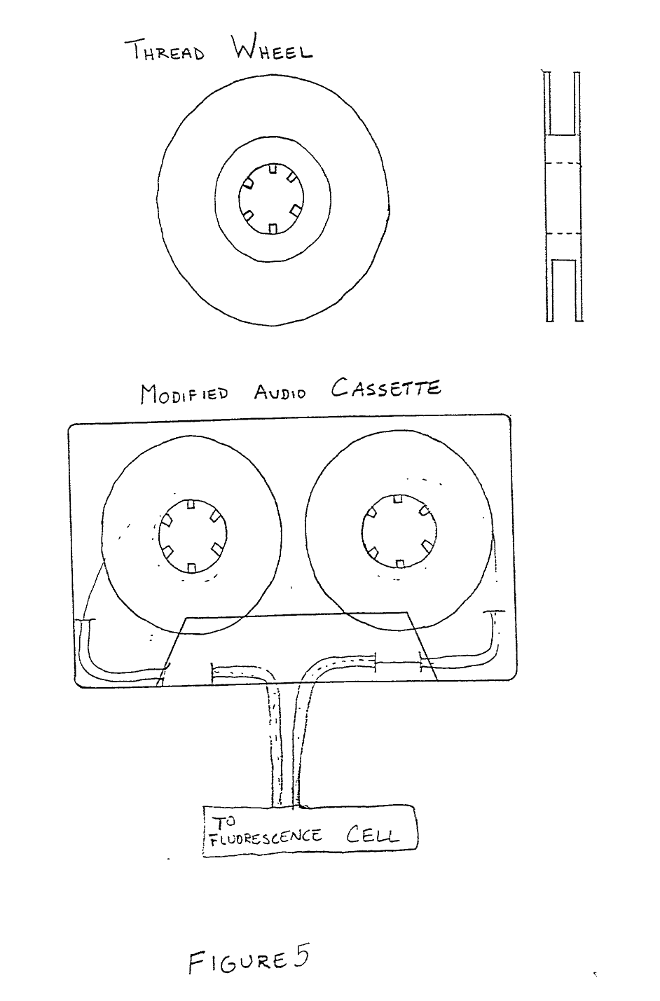

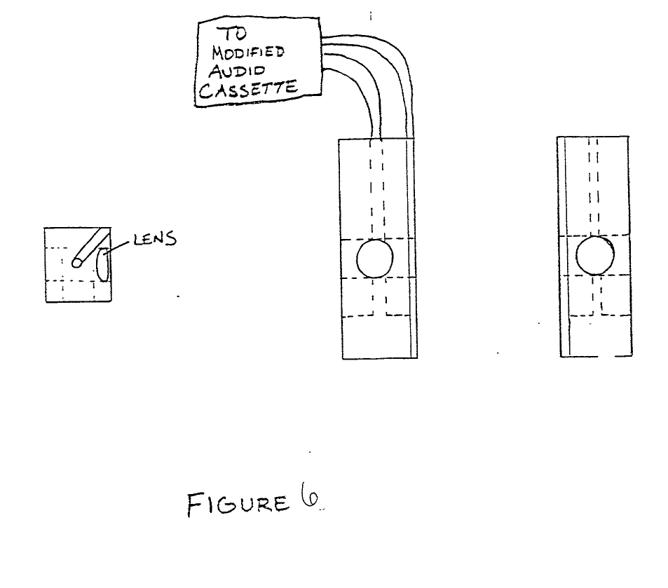

The fluorescein labeled thread library was mounted into the modified audio cassette, linked by teflon tubes to the fluorescent cell (FIG. 5). Once the library was in place, the thread was pulled through using the modified tape player while recording the PMT output via the A/D board hooked up through the computer. Library was analyzed by fluorescence spectrometer at 488 nm excitation and 535 nm emission. This reading was done several times so that any inconsistencies were determined directly. The fluorescence output signal in these experiments was read as a function of time. The region of thread which corresponds to each sample was easily determined because of the black wax used to separate each sample region. The thread itself had a slight fluorescence, so the evenly spaced negative deviations in the fluorescence signal due to black markings indicated divisions between samples. Fluorescence maxima were identified, checked to see that they were fairly evenly spaced and of the correct number, and then each maximum was recorded as its value above the minima on either side. Each peak in the data represents a separate compound, and since the absolute beginning of the library is known, the identity of each peak was determined simply by measuring the distance from the end of the library. Since this particular library was known to repeat after 35 compounds, it was possible to average the peaks on a repeat time of 35 (1 is averaged with 36, 71, etc. . . . ). This gives a more reliable value for each peak height. The resulting plot of peak height vs. distance along the thread is presented in FIG. 7.

The peaks in FIG. 7, going from left to right, represent the following series, in this order:

A1, B2, C3, D4, E5, A6, B7, etc.

Where:

| X1 | X2 |
|---|---|
| A = His | 1 = Ac Leu |
| B = Ser | 2 = Ac Phe |
| C = Asp | 3 = Bz |
| D = Ala | 4 = Ac |
| E = Phe | 5 = Ac His |
| | 6 = Ac Glu |
| | 7 = Ac Gly |

In this library, the expected highest peaks are those representing His in the final amino acid position (X₂-His-Pro-Gln-Phe-Ala-Ala-Ala-thread). The endcapping species should make less difference. Both of these expected results are seen.

Fourier Transformation Analysis

The data obtained from the thread reading was reduced to 2 points per compound, as outlined above (one point for each signal, taken as the average rise above the valley on either side of the signal, and one point between each peak). The Fourier transformation (FT) was done using a BASIC program using standard algorithms. In a preferred embodiment, the FT should be resonant: a radix 2 algorithm would be less appropriate, and less proper oversampling of data were ensured. The “waveform” corresponding to efficacy of particular amino acids installed on a given cylinder was extracted as follows. The real and imaginary parts of the peak at the relevant frequency were extracted from the frequency domain, as were all harmonics. These values were then put into a smaller array and Fourier transformed back to the time domain. The resulting “waveform” represents the output signal for each of the functional groups added on that cylinder. The signal for the 35 compound library, shown in FIG. 7, was Fourier transformed, and the waveforms corresponding to the 5 and 7 cm cylinders were extracted from the FT spectrum. These waveform binding profiles are shown in FIG. 8.

All references and patents cited herein are incorporated by reference in their entirety.

We claim:

1. An array of chemical compounds attached to a support, wherein each compound is attached to a pre-determined portion of the support.

2. The array of claim 1, prepared by a method which comprises the steps of:
providing a support having reactive functionalities;
subjecting said support to a set of reagents or reaction conditions, wherein each of said reagents or reaction conditions cycles with a specific period along the support, and wherein each individual reagent or reaction condition in the set is identified as a function of a unique distance or time; and
subjecting said support to one or more additional set of reagents or reaction conditions, wherein each of said reagents or reaction conditions cycles with a specific period along the support, and wherein each individual reagent or reaction condition in said one or more sets is identified as a function of unique distance or time, until a desired array of compounds is obtained.

3. The array of claim 1, prepared by a method which comprises the steps of:
a) providing a support having reactive functional groups,
b) winding the support around a geometric template,
c) dividing the surface of the template lengthwise into regions,
d) subjecting each region to one or more reagents or reaction conditions so as to attach reactive moieties or to modify the functional groups; and
e) repeating steps (b) through (d) until the desired library is obtained.

4. The array of claim 3, wherein the reactive moieties have additional functional groups which are masked by protecting groups, and wherein these protecting groups are removed prior to treatment with one or more reagents or reaction conditions.

5. The array of claim 1, wherein the identity of each compound in said array is uniquely specified by its location on the support.

6. The array of claim 1, wherein each of said compounds is synthesized from one or more reagents, and wherein each of said one or more reagents is added at a specific repeat frequency, defined at a specific location on the support.

7. The array of claim 1, wherein said array is one-dimensional.

8. A method of preparing an array of compounds comprising the steps of:
providing a support having reactive functionalities;
subjecting said support to a set of reagents or reaction conditions, wherein each of said reagents or reaction conditions cycles with a specific period along the support, and wherein each individual reagent or reaction condition in the set is identified as a function of a unique distance or time; and
subjecting said support to one or more additional set of reagents or reaction conditions, wherein each of said reagents or reaction conditions cycles with a specific period along the support, and wherein each individual reagent or reaction condition in said one or more sets is identified as a function of unique distance or time, until a desired array of compounds is obtained.

9. The method of claim 8, wherein said thread comprises a support consisting of a single material.

10. The method of claim 9, wherein said support comprises a single surface derivatized material.

11. The method of claim 8, wherein said support comprises a composite support.

12. The method of claim 8, wherein said support comprises a discontinuous synthesized support arrayed on a continuous structural support.

14. The method of claim 8, after the step of providing a support, further comprising:
winding the support around a geometric template; and
dividing the surface of the geometric template into parallel regions.

15. The method of claim 14, wherein said support comprises a geometric template selected from the group consisting of cylinder, prism of polygonal cross section, cylinder with ridges to distinguish regions, flat plate, and conic section.

16. The method of claim 8, wherein the linear array of compounds comprises an array of compounds comprising a contiguous portion of a linear sequence of compounds and represents an optimally diverse subset.

17. The method of claim 8, wherein the linear array of compounds comprises an array of compounds synthesized from a support longer than necessary to produce a single copy of each library member, and thus provides a set of duplicates to evaluate reproducibility.

18. The method of claim 8, wherein the step of providing a linear array of compounds comprises providing an array of compounds in which each possible combination is represented once.

19. A method of preparing a chemical array, which comprises the steps of
a) providing a support having reactive functional groups,
b) winding the support around a geometric template,
c) dividing the surface of the template lengthwise into regions,
d) subjecting each region to one or more reagents or reaction conditions so as to attach reactive moieties or to modify the functional groups; and
e) repeating steps (b) through (d) until the desired library is obtained.

20. The method of claim 19, wherein the reactive moieties have additional functional groups which are masked by protecting groups, and wherein these protecting groups are removed prior to treatment with one or more reagents or reaction conditions.

21. The method of claim 19, wherein said support comprises a geometric template selected from the group consisting of cylinder, octagon, hexagon, rectangle, and cylinders with ridges to distinguish regions.

22. The method of claim 19, wherein the linear array of compounds comprises an array of compounds comprising a contiguous portion of a linear sequence of compounds and represents an optimally diverse subset.

23. The method of claim 19, wherein the linear array of compounds comprises an array of compounds synthesized from a support longer than necessary to produce a single copy of each library member, and thus provides a set of duplicates to evaluate reproducibility.

24. The method of claim 19, wherein the linear array of compounds comprises an array of compounds in which each possible combination is represented once.

25. A method of measuring a property of each of the chemical compounds in an array comprising the steps of:
providing a linear array of chemical compounds, such that the identity of each of the compounds is a function of distance or time with respect to the start of the array;
assaying compounds in an array to detect those compounds having a specific desired activity; and
transporting said linear array of compounds at a constant velocity through an appropriate detector capable of detecting compounds having a specific desired activity.

26. The method of claim 25, wherein each of the compounds is attached to a support.

27. The method of claim 26, wherein each of the compounds is assayed while attached to the support.

28. The method of claim 25, wherein each of the compounds is cleaved from the support prior to the step of assaying.

29. The method of claim 25, wherein the linear array of compounds comprises an array of compounds comprising a contiguous portion of a linear sequence of compounds and represents an optimally diverse subset.

30. The method of claim 25, wherein the linear array of compounds comprises an array of compounds synthesized from a support longer than necessary to produce a single copy of each library member, and thus provides a set of duplicates to evaluate reproducibility.

31. The method of claim 25, wherein the linear array of compounds comprises an array of compounds in which each possible combination is represented once.

32. A method of assaying chemical compounds for binding to fluorescent species comprising:
preparing an array of compounds on a linear optical fiber;
contacting said array of compounds in solution with fluorescent species;
exciting said fluorescent species by providing a light source; and
detecting specific library members capable of binding to fluorescent species.

33. The method of claim 32, wherein the steps of exciting said fluorescent species and detecting specific library members comprises an apparatus capable of simultaneously providing a light source and moving said support at a constant rate through the apparatus, so as to identify the distance or time at which specific compounds that are capable of binding occur, and thereby to identify the identity of the specific compound.

34. A method of obtaining structure-activity relationships from the compounds in a library, which comprises the steps of:
providing a linear array of compounds,
measuring the activity of each compound in the library, so as to obtain a datapoint for each compound,
arranging the datapoints in a linear array, in such a way that variable structural features in the library are repeated at fixed intervals in the array, and
mathematically processing the resulting linear array of datapoints by Fourier transformation.

35. The method of claim 34, wherein the step of providing a linear array of compounds comprises providing an array of compounds comprising a contiguous portion of a linear sequence of compounds and represents an optimally diverse subset.

36. The method of claim 34, wherein the step of providing a linear array of compounds comprises providing an array of compounds synthesized from a support longer than necessary to produce a single copy of each library member, and thus provides a set of duplicates to evaluate reproducibility.

37. The method of claim 34, wherein the step of providing a linear array of compounds comprises providing an array of compounds in which each possible combination is represented once.
